(19) United States
(12) Patent Application Publication
(10) Pub. No.: US 2023/0285620 A1
(43) Pub. Date: Sep. 14, 2023

(54) STERILIZATION APPARATUS AND ASSOCIATED CONTAINER

(71) Applicant: IDEATE Medical Inc., Town & Country, MO (US)

(72) Inventors: Philippe Conseil, Roxboro (CA); Florencia Chicatun, Saint-Laurent (CA); William Wong, Encinitas, CA (US)

(73) Assignee: IDEATE Medical Inc., Town & Country, MO (US)

(21) Appl. No.: 18/168,706

(22) Filed: Feb. 14, 2023

Related U.S. Application Data
(60) Provisional application No. 63/317,684, filed on Mar. 8, 2022.

(51) Int. Cl. A61L 2/26; A61L 2/18

(52) U.S. Cl. CPC A61L 2/26; A61L 2/18; A61L 2202/122

(57) ABSTRACT

A sterilization system and associated components and methods for sterilizing an article. The sterilization system includes a container for use with a sterilization apparatus for sterilizing the article. The container includes an exterior fluid connector port facing an environment surrounding the container that is fluidly connectable to a first fluid connector of the sterilization apparatus. An interior fluid aperture face is fluidly coupled to the exterior fluid connector port and fluidly connects to the article. A valve is fluidly disposed between the exterior fluid connector port and the interior fluid aperture. The valve is arranged to be engaged by the sterilization apparatus to move a valve member to an open position to permit fluid flow between the first fluid connector and the article when the sterilization container is used with the sterilization apparatus and when the article is fluidly connected to the interior fluid aperture.

### CROSS-REFERENCE TO RELATED APPLICATION

This application claims priority to U.S. Provisional App. No. 63/317,684, filed on Mar. 8, 2022, the entirety of which is hereby incorporated by reference.

### FIELD

The present disclosure generally relates to sterilization systems, and, more specifically, to a sterilization apparatus and associated container for sterilizing one or more articles.

### BACKGROUND

Certain articles, such as medical devices (e.g., endoscopes), need to be sterilized between uses. These articles can include interior lumens, which require sterilization. One way to sterilize these interior lumens is to move a sterilization fluid through the lumens. For example, PCT Publication No. WO 2018/090133 describes a sterilization system where an endoscope is placed within a chamber with a port connector attached to a fluid port of the endoscope. The port connector fluidly couples the interior lumen(s) of the endoscope with a pressure source creating a pressure differential. To sterilize the interior lumen(s) of the endoscope, a sterilization fluid (such as hydrogen peroxide vapor) is introduced into the chamber and then drawn through the interior lumen(s) of the endoscope via the pressure differential created by the pressure source.

### SUMMARY

In one aspect, a sterilization container for use with a sterilization apparatus for sterilizing an article comprises a body defining an interior configured to receive the article. An exterior fluid connector port faces an environment surrounding the body. The exterior fluid connector port is fluidly connectable to a first fluid connector of the sterilization apparatus. An interior fluid aperture faces the interior of the body and is in fluid communication with the interior of the body. The interior fluid aperture is configured to be fluidly connected to the article. The interior fluid aperture is fluidly coupled to the exterior fluid connector port. A valve is fluidly disposed between the exterior fluid connector port and the interior fluid aperture. The valve includes a valve member movable to an open position in which the valve permits fluid flow between the exterior fluid connector port and the interior fluid aperture and to a closed position in which the valve blocks fluid flow between the exterior fluid connector port and the interior fluid aperture. The valve is arranged to be engaged by the sterilization apparatus to move the valve member to the open position to permit fluid flow between the first fluid connector and the article when the sterilization container is used with the sterilization apparatus and when the article is fluidly connected to the interior fluid aperture.

In another aspect, a sterilization apparatus for sterilizing an article within a sterilization container comprises a sterilization chamber defining an interior sized and shaped to receive the sterilization container. A container connector assembly is configured to fluidly connect the sterilization container to the sterilization apparatus. The container connector assembly includes a fluid connector movable between a retracted position and an extended position. The fluid connector is configured to engage the sterilization container when the fluid connector moves from the retracted position toward the extended position to fluidly connect the fluid connector to the sterilization container.

In another aspect, a sterilization system for sterilizing an article comprises a sterilization container configured to receive the article. A sterilization chamber defines an interior sized and shaped to receive the sterilization container. A fluid connector is configured to fluidly connect the sterilization container to the sterilization apparatus. At least one of the fluid connector or the sterilization container is configured to move toward the other of the fluid connector or the sterilization container to fluidly connect the fluid connector and the sterilization container to one another.

In another aspect, a sterilization container for use with a sterilization apparatus for sterilizing an article comprises a body defining an interior configured to receive the article. An exterior fluid connector port faces an environment surrounding the body. The exterior fluid connector port is fluidly connectable to a first fluid connector of the sterilization apparatus. An interior fluid aperture faces the interior of the body and is in fluid communication with the interior of the body. The interior fluid aperture is configured to be fluidly connected to the article. The interior fluid aperture is fluidly coupled to the exterior fluid connector port. A valve is fluidly disposed between the exterior fluid connector port and the interior fluid aperture. The valve includes a valve member movable to an open position in which the valve permits fluid flow between the exterior fluid connector port and the interior fluid aperture and to a closed position in which the valve blocks fluid flow between the exterior fluid connector port and the interior fluid aperture. A valve actuator is operatively coupled to the valve member. The valve actuator is arranged to be engaged by the sterilization apparatus to move the valve member toward the open position to permit fluid flow between the first fluid connector and the article when the sterilization container is used with the sterilization apparatus and when the article is fluidly connected to the interior fluid aperture.

Other objects and features will be in part apparent and in part pointed out hereinafter.

### BRIEF DESCRIPTION OF THE DRAWINGS

FIG. 1 is a schematic illustration of a sterilization system including a sterilization apparatus according to one embodiment of the present disclosure;

FIG. 2 is front perspective of a sterilization container of the sterilization system;

FIG. 3 is a rear perspective of the sterilization container;

FIG. 4 is a perspective of a box of the sterilization container;

FIG. 5 is a rear perspective of a bulkhead of the sterilization container;

FIG. 6 is a front perspective of the bulkhead;

FIG. 7 is a longitudinal cross-section of the bulkhead;

FIG. 8 is a perspective of a sterilization chamber of the sterilization apparatus with the sterilization container therein;

FIG. 9 is a schematic cross-sectional illustration of the sterilization chamber with the sterilization container therein;

FIG. 10 is a schematic side view illustration of a container connector assembly of the sterilization chamber;

FIG. 11 is a schematic top view illustration of the container connector assembly;

FIG. 12 is an enlarged schematic side view illustration of the sterilization container and the container connector assembly, the container connector assembly shown in a retracted position;

FIG. 13 is similar to FIG. 12 but the container connector assembly is shown in an extended position; and

FIG. 14 is schematic longitudinal cross-sectional illustration of the sterilization container with the container connector assembly shown in the extended position.

Corresponding reference characters indicate corresponding parts throughout the drawings.

### DETAILED DESCRIPTION

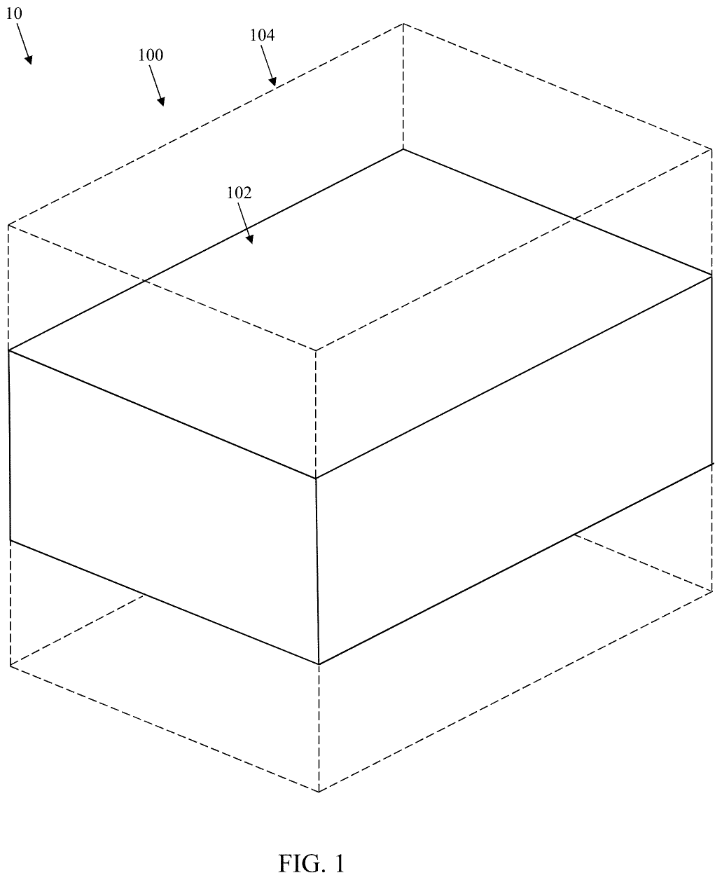

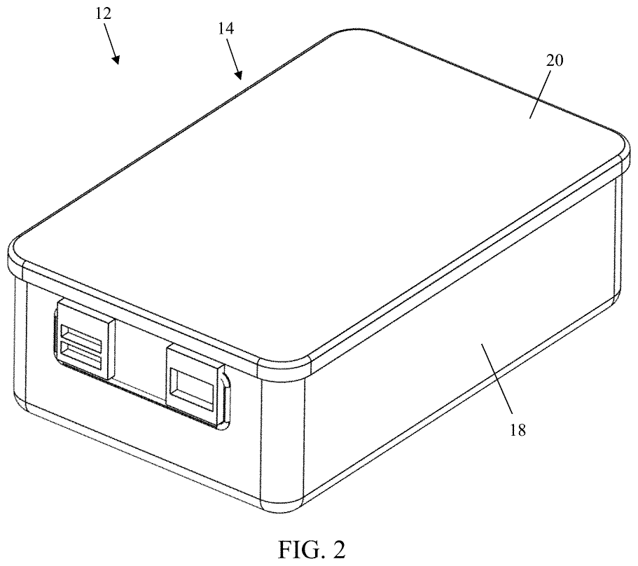

Referring to FIGS. 1 and 2, one embodiment of a sterilization apparatus (e.g., a sterilizer) according to the present disclosure is generally indicated by reference numeral 100 and one embodiment of a sterilization container according to the present disclosure is generally indicated at 12. Collectively, the sterilization apparatus 100 and the sterilization container 12 form a sterilization system, generally indicated at 10, for sterilizing one or more articles (not shown), such as endoscopes. The sterilization container 12 holds the articles and is placed in the sterilization apparatus 100, which then sterilizes the articles using a sterilization fluid (e.g., a sterilant), such as hydrogen peroxide vapor. It will be understood that the use of other suitable sterilants are also within the scope of the present disclosure. The sterilization apparatus 100 generally draws (broadly, moves) the sterilization fluid over the articles and through one or more interior lumens of the articles to sterilize the articles. Further details on how the sterilization apparatus 100 sterilizes articles may be found in PCT Publication No. WO 2018/090133.

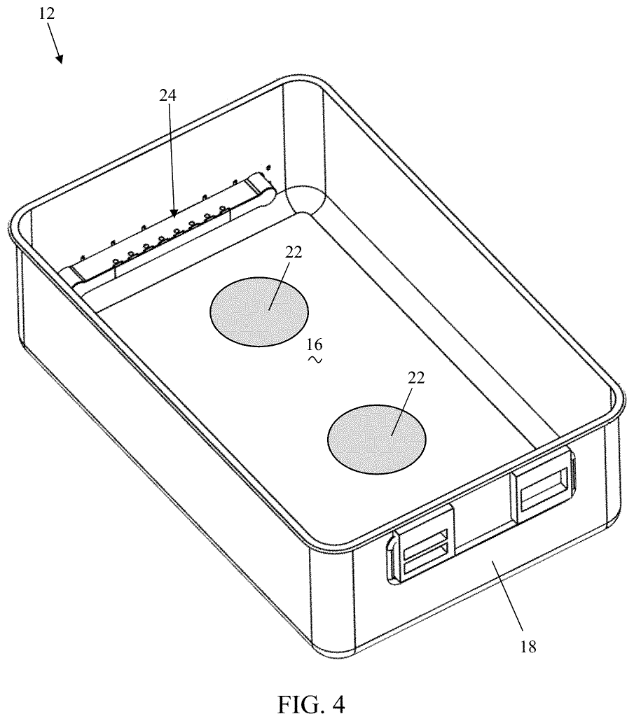

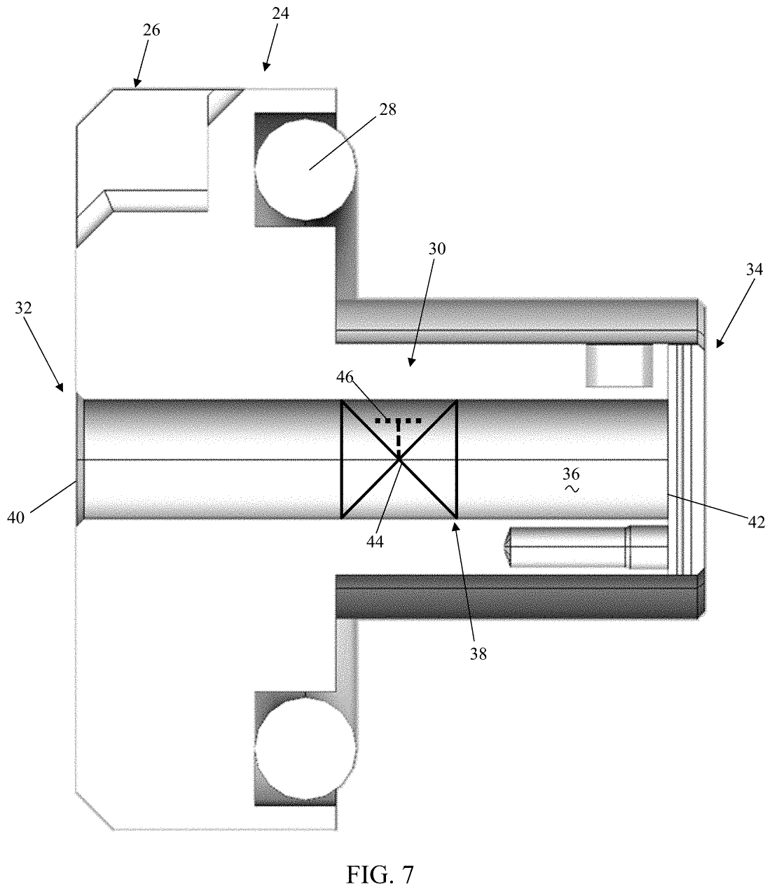

Referring to FIGS. 2-7, the sterilization container 12 is configured to hold one or more articles that are to be sterilized by the sterilization apparatus 100. The sterilization container 12 is used with the sterilization apparatus 100 to sterilize the articles. More specifically, the sterilization container 12 is configured to be located within and connected to the sterilization apparatus 100 for receiving the sterilization fluid delivered into the sterilization apparatus into an interior 16 of the sterilization container to sterilize articles held in the sterilization container. The sterilization container 12 includes a body 14 that defines the interior 16 configured to receive the one or more articles. The body 14 includes a base or box 18 and a lid 20. The lid 20 is attachable to the box 18 and forms a fluid tight seal with the box to prevent fluid (e.g., gas, liquid, vapor, etc.) from passing therebetween. Removing the lid 20 from the box 18 allows the articles to be placed in or removed from the interior 16. The box 18 has a base with opposite front and rear end walls extending upward from the base and opposite left and right side walls extending upward from the base. The sterilization container 12 may include one or more latches or clasps (not shown) to secure the lid 20 to the box 18. The sterilization container 12 includes one or more sterilant permeable membranes 22. These membranes 22 can be generally anywhere on the box 18 and/or lid 20. In the illustrated embodiment, the membranes 22 are attached to the base of the box 18. Each membrane 22 is configured to permit the sterilization fluid in the environment surrounding the body 14 (e.g., the environment outside the sterilization container 12) to pass therethough and into the interior 16 of the body. The sterilization fluid is drawn through the membranes 22 by the sterilization apparatus 100, as generally described in PCT Publication No. WO 2018/090133, in order to sterilize the articles contained in the interior 16. The membranes 22 are preferably contaminant (e.g., dust, debris, microorganism, etc.) impermeable to prevent any egress of contaminants into the interior 16 of the sterilization container 12. This helps maintain the sterility of the interior 16 and articles contained therein after the articles have been sterilized.

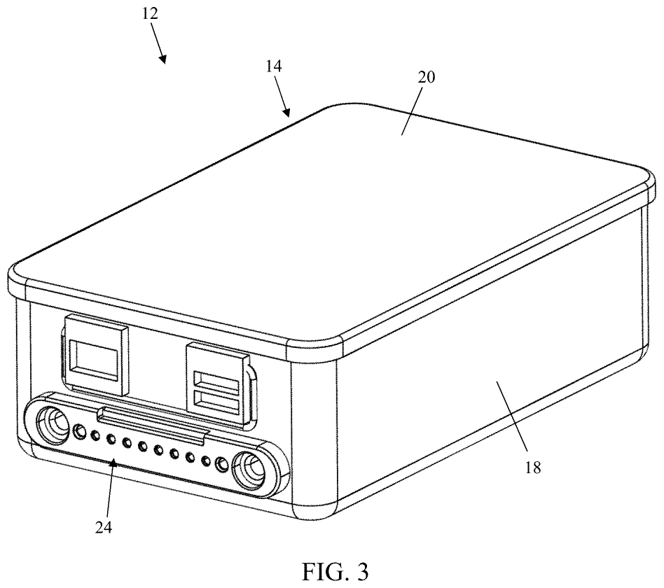

Referring to FIGS. 3-6, the sterilization container 12 includes a bulkhead 24 mounted on the box 18. As will become apparent, the bulkhead 24 is used to fluidly connect the interior 16 of the sterilization container 12, specifically the articles contained in the interior, with the sterilization apparatus 100 to allow the sterilization apparatus to draw the sterilization fluid into and out of the interior. The bulkhead 24 extends through the rear wall of the box 18. The bulkhead 24 includes a body 26 and a seal or gasket 28. The seal 28 is disposed between the body 26 and the rear wall of the box 18 to form a fluid tight seal to prevent fluid from passing between the body and the box. The seal 28 generally extends around the outer perimeter of the body 26.

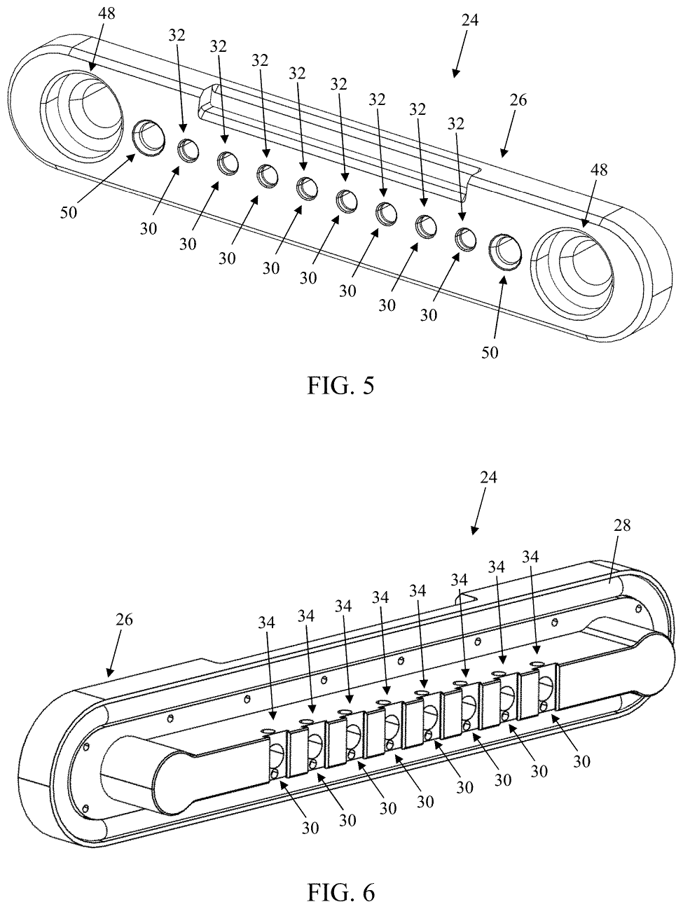

Referring to FIGS. 5-7, the bulkhead 24 includes a plurality of article plumbings 30. Each article plumbing 30 is configured to fluidly couple to a corresponding article at one end and to the sterilization apparatus 100 at another end to fluidly couple the article to the sterilization apparatus 100. This way, the sterilization apparatus 100 can move the sterilization fluid through the article via the associated article plumbing 30. Generally, each article plumbing 30 will be associated with one article, although other arrangements (e.g., two or more articles associated with one article plumbing) are within the scope of the present disclosure. In the illustrated embedment, the sterilization container 12 is configured to sterilize eight different articles, so the bulkhead 24 includes eight article plumbings 30, one or each article. More or fewer article plumbings 30 are within the scope of the present disclosure. The article plumbings 30 are generally identical so one article plumbing will now be described with the understanding that the description applies to all the article plumbings.

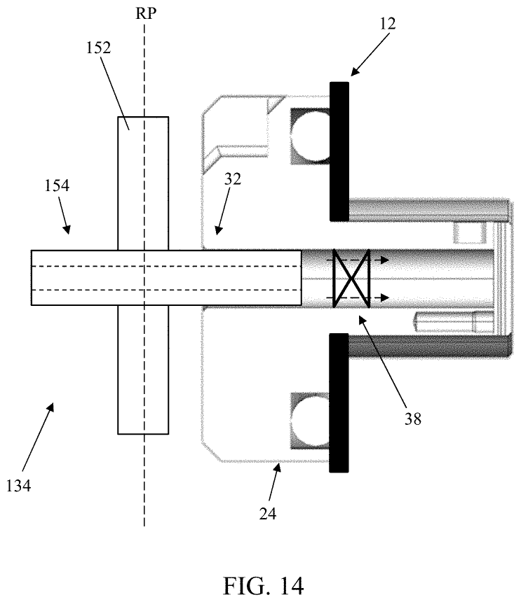

Referring to FIG. 7, the article plumbing 30 (broadly, the sterilization container 12) includes an exterior fluid connector port 32, an interior fluid connector port 34, passaging 36 extending between the ports, and a valve 38 (broadly, a selectively fluid permeable member) disposed in the passaging. Therefore, the exterior and interior fluid connector ports 32, 34 are fluidly coupled together. The passaging 36 fluidly couples the exterior and interior fluid connector ports 32, 34. The exterior fluid connector port 32 is connectable (e.g., fluidly connectable) to a fluid connector 154 (FIG. 14) of the sterilization apparatus 100. The exterior fluid connector port 32 is generally disposed on the exterior of the body 14 and faces the environment surrounding the body (e.g., faces exteriorly). In the illustrated embodiment, the exterior fluid connector port 32 includes an exterior fluid aperture 40. The exterior fluid aperture 40 is sized and shaped to receive the fluid connector 154. Specifically, the exterior fluid aperture 40 permits the fluid connector 154 to extend therethrough and into the passaging 36 to allow the fluid connector to engage the valve 38, as described in more detail below. The interior fluid connector port 34 is connectable (e.g., fluidly connectable) to an article connector (not shown) associated with the article for fluidly connecting the article (specifically, the one or more interior lumens thereof) thereto. Generally, the article connector can be any suitable connector for connecting the article to the article plumbing 30. In some embodiment, the article plumbing 30 may include the article connector. In other embodiment, the article connector may be omitted and the article can be directly connected to the interior fluid connector port 34. In the illustrated embodiment, the interior fluid connector port 34 includes a dovetail slot for connecting to the article connector. The interior fluid connector port 34 is generally disposed on the interior side of the body 14. The interior fluid connector port 34 faces the interior 16 of the body 14 and is in fluid communication with the interior of the body. In the illustrated embodiment, the interior fluid connector port 34 includes (e.g., defines) an interior fluid aperture 42. The interior fluid aperture 42 is configured to be fluidly connected to the article. Specifically, the interior fluid aperture 42 is fluidly connected to the article (e.g., the interior lumen(s) thereof) when the article connector connects the article to the interior fluid connector port 34. Other configurations of the exterior and interior connector ports are within the scope of the present disclosure.

The valve 38 is fluidly disposed between the exterior and interior fluid connector ports 32, 34 (e.g., the exterior and interior fluid apertures 40, 42) to selectively block or permit fluid flow (e.g., the flow of the sterilization fluid) therebetween. The valve 38 is generally disposed in the passaging 36 between the exterior and interior fluid connector ports 32, 34. The valve 38 includes a valve member 44 (FIG. 7). The valve member 44 is movable between a closed position (FIG. 7) and an open position (not shown). When the valve member 44 is in the closed position, the valve 38 is closed such that it blocks or prevents the flow of any fluid, such as the sterilization fluid, between the exterior and interior fluid connector ports 32, 34. When the valve member 44 is in the open position, the valve 38 is open such that it permits the flow of any fluid, such as the sterilization fluid, between the exterior and interior fluid connector ports 32, 34. The valve member 44 is biased toward the closed position, such as by a spring (not shown). Other selectively fluid permeable members, besides valves, that can permit the sterilant to pass through and prevent the egress of contaminants into the interior 16 of the sterilization container 12 are within the scope of the present disclosure. For example, in one embodiment, the selectively fluid permeable member comprises a membrane, similar to the membrane 22 described above, that permits sterilant to move through the article plumbing but inhibits the egress of contaminants.

In the illustrated embodiment, the valve member 44 is configured to be operatively connected to the sterilization apparatus 100 so that the sterilization apparatus can move the valve member toward the open position. In the illustrated embodiment, the valve 38 is arranged to be engaged by the sterilization apparatus 100 to move the valve member 44 to the open position to permit fluid flow between the exterior and interior fluid connector ports 32, 34. This way, the sterilization apparatus 100 places the valve member 44 in the open position when the sterilization container 12 is used with the sterilization apparatus to enable the sterilization apparatus to move the sterilization fluid through the article plumbing 30 (e.g., the valve). In operation, when the sterilization container 12 is used with (e.g., connected to) the sterilization apparatus 100 and when the article is fluidly connected to the interior fluid connector port 34, placing the valve member 44 in the open position permits fluid flow between the fluid connector 154 of the sterilization apparatus (connected to the exterior fluid connector port 32) and the article. This allows the sterilization apparatus 100 to sterilize the article by moving the sterilization fluid through the article via the article plumbing 30 of the sterilization container 12. In the illustrated embodiment, the valve 38 is arranged to be engaged by the fluid connector 154 of the sterilization apparatus 100 to move the valve member to the open position. A valve actuator 46 is operatively coupled to the valve member 44. Actuating the valve actuator moves the valve member 44 between the open and closed positions to open and close the valve. In the illustrated embodiment, the valve actuator 46 is part of the valve 38, although it is understood the valve actuator may be separate from the valve. In one embodiment, the valve actuator 46 is a push member or plate disposed in the passaging 36 on the exterior port connector 32 side of the valve. In this embodiment, the valve actuator 46 is arranged to be engaged and pushed by the end of the fluid connector 154 of the sterilization apparatus 100 to move the valve member 44 toward the open position when the fluid connector is connected to the exterior fluid connector port 32 (e.g., when the fluid connector is inserted through the exterior fluid aperture 40 and into the passaging 36). The fluid connector 154 of the sterilization apparatus 100 continues to engage the valve 38 to hold the valve member 38 in the open position while the sterilization container 12 is connected to (e.g., mated with) the sterilization apparatus 100 to permit the sterilization fluid to flow between the interior 16 and the sterilization apparatus. Because the valve member 44 is biased toward the closed position, when the fluid connector 154 of the sterilization apparatus 100 is removed from the exterior fluid connector port 32, the fluid connector disengages the valve actuator 46 of the valve 38, thereby allowing the valve member 44 to move to the closed position. Accordingly, when the sterilization container 12 is disconnected from the sterilization apparatus 100, the valve 38 automatically closes to seal the interior 16 and the articles contained therein from the outside environment to maintain the sterility of the articles. As long as the sterilization container 12 is not opened by removing the lid 20, the interior 16 and the articles sterilized therein will remain sterile and generally free of outside contaminants for an extended period of time, such as months or years. This allows the sterilization container 12 to be used to sterilize the articles and then the sterilization container can be stored, such as on a self, until such time as the articles contained therein are needed by a user. When the articles are needed, the user simply removes the lid 20 and removes the still sterile articles from the interior 16 of the sterilization container 12. In the illustrated embodiment, the valve actuator 46 is part of the valve. In other embodiments, the valve actuator may be separate from the valve and operatively connected to the valve member, such as by a linkage, such that actuation of the valve actuator results in corresponding actuation of the valve member. In addition, the valve actuator can be disposed at other locations on the container 12 and/or engaged by other components of the sterilization apparatus 100 to open and close the valve 38. For example, the valve actuator can be disposed on a wall (e.g., rear wall of the box 18). In addition, other ways of actuating the valve actuator, besides engagement with the sterilization apparatus, are within the scope of the present disclosure. For example, the valve actuator can be manually actuated. In another example, the valve actuator can be actuated by a prime mover, such as an electric motor, a linear actuator, solenoid, or the like. In another example, the valve actuator may comprise the prime mover.

Referring to FIG. 5, the bulkhead 24 (broadly, the sterilization container 12) includes several features to facilitate the connection or matting of the bulkhead (broadly, the sterilization container) with the sterilization apparatus 100. In the illustrated embodiment, the bulkhead 24 includes two initial alignment guides 48 (broadly, at least one initial alignment guide) and two final alignment guides 50 (broadly, at least one final alignment guide). Each initial alignment guide 48 is configured to be engaged by the sterilization apparatus 100 (specifically, an initial alignment guide 140 thereof) to initially position the sterilization container 12 relative to the sterilization apparatus. In the illustrated embodiment, each initial alignment guide 48 includes a blind hole. The blind hole is arranged, sized and shaped to receive a corresponding initial alignment guide 140 of the sterilization apparatus 100. The blind hole may be defined by one or more tapered surfaces to help move the sterilization container 12 and sterilization apparatus 100 relative to one another. In addition, each final alignment guide 50 is configured to be engaged by the sterilization apparatus 100 (specifically, a final alignment guide 162 thereof) to align the exterior fluid connector ports 32 and the corresponding fluid connectors 154 of the sterilization apparatus 100 with each other. In the illustrated embodiment, each final alignment guide 50 includes a blind hole. The blind hole of the final alignment guide 50 has a much smaller cross-sectional area than the blind hole of the initial alignment guide 48. The blind hole of the final alignment guide 50 is arranged, sized and shaped to receive a corresponding final alignment guide 162 of the sterilization apparatus 100. Generally, the initial alignment guides 48 provide the initial, coarse positional adjustment between the sterilization container 12 and the sterilization apparatus 100 while the final alignment guides 50 provide the final, fine alignment between the exterior fluid connector ports 32 and the fluid connectors 154 of the sterilization apparatus 100, as described in more detail below.

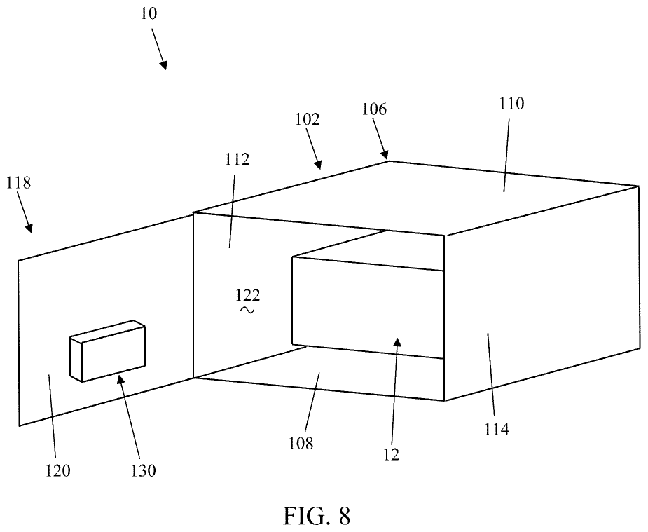

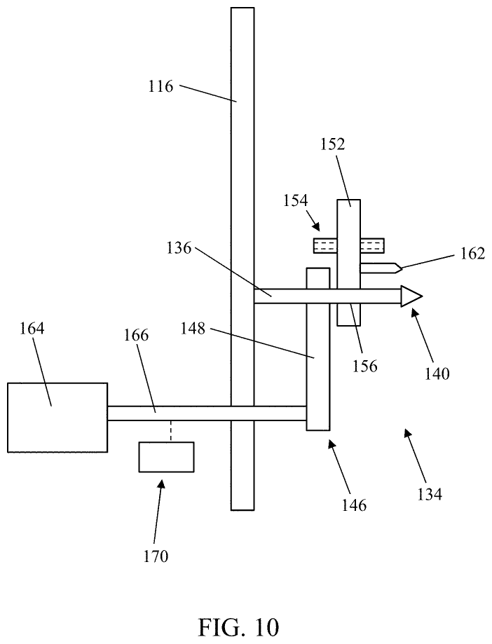

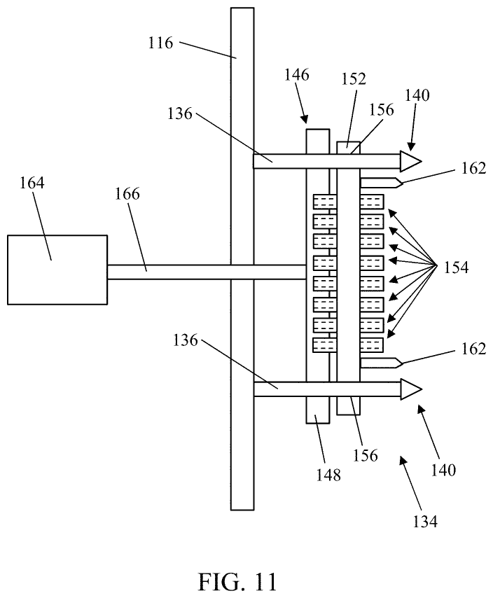

Referring to FIGS. 1 and 8-14, the sterilization apparatus 100 is used to sterilize the one or more articles 100 within the sterilization container 12. The sterilization apparatus 100 includes a sterilization chamber 102 and other components (not shown), such as tanks, pumps, plumbing, controls, etc., supported by a housing, generally indicated at 104 (FIG. 1). For the purposes of this disclosure, generally only the sterilization chamber 102 and corresponding associated components are shown. Further details on the other components of the sterilization apparatus 100 not shown may be found in PCT Publication No. WO 2018/090133. In the illustrated embodiment, the sterilization apparatus 100 only includes one sterilization chamber 102. However, it is understood that the sterilization apparatus 100 can include more than one (e.g., two, three, four, etc.) sterilization chambers 102.

The sterilization chamber 102 includes a housing 106 having a bottom wall 108, a top wall 110, opposite left and right side walls 112, 114, a rear wall 116 and a door 118 defining a front wall 120. The housing 106 defines an interior 122 sized and shaped to receive the sterilization container 12. The interior 122 has an open front the door 118 closes and through which the sterilization container 12 can be inserted into and removed from the interior. The sterilization chamber 102 may include one or more bottom guide rails above the bottom wall 108 along which the sterilization container 12 can slide. These guide rails may also space the bottom of the sterilization container 12 from the bottom wall 108 to allow the sterilization fluid to move through the membranes 22 in the bottom of the sterilization container. The sterilization chamber 102 may include one or more (e.g., two) side or lateral guides or guide rails to laterally position the sterilization container 12 within the interior 122. In one embodiment, the lateral guides are arranged to engage the sides of the sterilization container 12 to laterally position the container. The sterilization chamber 102 may also include a stop 130 (e.g., a front stop) to limit the longitudinal movement (e.g., forward movement) of the sterilization container 12 within the interior 122 for reasons that will become apparent. In the illustrated embodiment, the stop 130 is supported by the door 118. The stop may include one or more resiliently compressible members (e.g., pins) arranged to engage the front of the sterilization container 12. Other configurations of the stop are within the scope of the present disclosure.

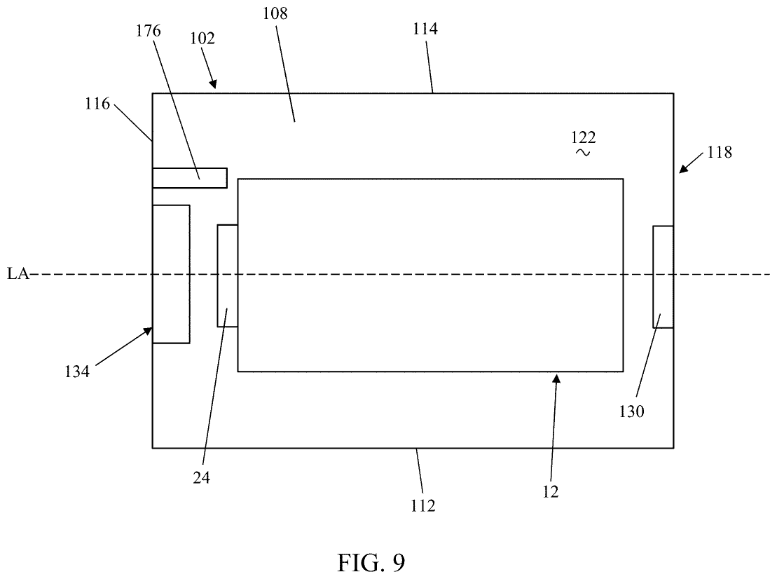

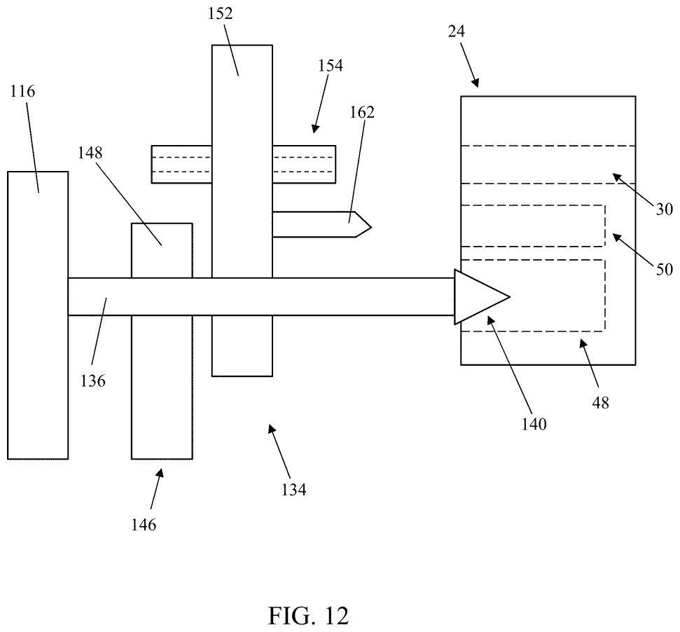

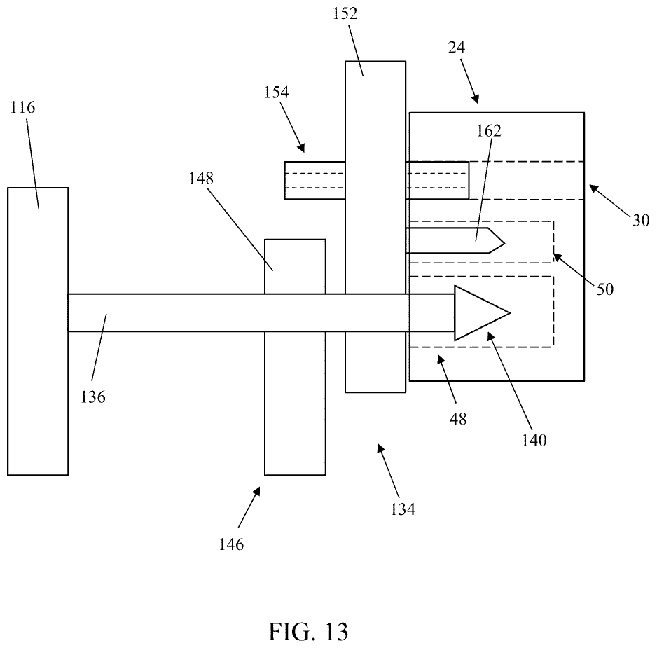

Referring to FIGS. 9-14, the sterilization apparatus 100 includes a container connector assembly 134 associated with the sterilization chamber 102. The container connector assembly 134 is configured to fluidly connect the sterilization container 12 to the sterilization apparatus 100. When the container connector assembly 134 is connected to or mated with the sterilization container 12 (specifically, the bulkhead 24 thereof), the sterilization apparatus 100 can draw the sterilization fluid into the interior 16 of the sterilization container 12 and through the one or more articles. The container connector assembly 134 is movable between a retracted position (FIG. 12) and an extended position (FIG. 13). In the retracted position, the container connector assembly 134 may be disconnected from (e.g., spaced apart from) and not fluidly connected to the sterilization container 12. In the extended position, the container connector assembly 134 is connected to or mated with the sterilization container 12 and fluidly coupled to the sterilization container 12. The container connector assembly 134 generally moves along a longitudinal axis LA of the sterilization chamber 102 between the retracted and extended positions. The longitudinal axis LA extends between the door 118 and the rear wall 116.

The container connector assembly 134 is movably (e.g., slidably) mounted on two slide rails or rods 136 (broadly, at least one slide rail) of the sterilization chamber 102. The slide rails 136 are elongate and generally cylindrical. The slide rails 136 are fixed to the rear wall 116 of the sterilization chamber 102 and extend forward toward the door 118. In one embodiment, the container connector assembly 134 includes two slides or sleeves (broadly, at least one slide), each mounted on one of the slide rails 136. Each slide can move or slide longitudinally along the length of its corresponding slide rail 136. Each slide may be elongate and generally cylindrical. Each slide may define a longitudinal cavity, with an open rear end, that receives the slide rod 136 and permits the slide to move relative to the slide rod. The container connector assembly 134 includes two (broadly, at least one) initial alignment guide 140. In one embodiment, the front end portion of each slide forms (e.g., defines) an initial alignment guide 140. Each initial alignment guide 140 of the container connector assembly 134 is configured to engage the sterilization container to initially position the sterilization container relative to the sterilization chamber 102 and the container connector assembly. Specifically, each initial alignment guide 140 of the container connector assembly 134 is configured to mate with (e.g., be inserted into) a corresponding one of the initial alignment guides 48 of the sterilization container 12, as shown in FIG. 13. In one embodiment, each initial alignment guide 140 of the container connector assembly 134 includes a generally conical tip arranged to be inserted into the blind hole of the corresponding one of the initial alignment guides 48 of the sterilization container 12. The conical tip may include a passageway between the longitudinal cavity and the environment outside the tip to relieve any pressure buildup or loss in the longitudinal cavity as the slide moves along the slide rail 136. In addition, each initial alignment guide 140 may include a circumferential flange rearward of the conical tip and arranged to engage the sterilization container 12 (specifically, the bulkhead 24) to limit the distance the initial alignment guide can be inserted into the blind hole of the initial alignment guides 48 of the sterilization container 12.

The container connector assembly 134 includes a pusher 146 movably (e.g., slidably) mounted on the two slides, and therefore by extension the two slide rails 136. Accordingly, the pusher 146 can move relative to slides, which can themselves move relative to their respective slide rails 136. The pusher 146 includes a push member or plate 148 and two slide bearings (not shown) secured to the plate. Each slide bearing is mounted on one of the slides, thereby permitting the pusher 146 to move (e.g., slide) relative to the slides. The container connector assembly 134 includes a fluid connector mount or plate 152. Mounted on the fluid connector mount 152 are a plurality (broadly, at least one) fluid connectors 154. In the illustrated embodiment, the container connector assembly 134 includes eight fluid connectors 154, although more or fewer fluid connectors are within the scope of the present disclosure. Preferably, the container connector assembly will include the same number of fluid connectors as the sterilization container 12 includes exterior fluid connector ports 32. The fluid connectors 154 are arranged on the fluid connector mount 152 to each be inserted into a corresponding exterior fluid connector port 32 of the sterilization container 12, as described herein. Each fluid connector 154 is moveable between the retracted position and the extended position, along the longitudinal axis LA. In the illustrated embodiment, all the fluid connectors 154 move together between the retraced and extended positions. Each fluid connector 154 is configured to engage the sterilization container 12 (e.g., mate with its corresponding exterior fluid connector port 32) when the fluid connector moves from the retracted position toward the extended position to fluidly connect the fluid connectors to the sterilization container. Each fluid connector 154 includes a forward insertion portion configured to be inserted into one of the exterior fluid apertures 40 of a corresponding exterior connector port 32 and a rearward conduit connection portion configured to be connected to a flexible fluid conduit (not shown) of the sterilization apparatus 100. The insertion portion of each fluid connector 154 may include a seal, such as an O-ring, that engages the interior surface of the bulkhead 24 that defines the passaging 36 to form a fluid tight seal between the fluid connector and the sterilization container 12.

The pusher 146 is configured to move the fluid connectors 154 from the retracted position toward the extended position. In particular, the pusher 146 is operatively connected to the fluid connector mount 152 to move the fluid connector mount and the fluid connectors 154 from the retracted position toward the extended position.

The fluid connector mount 152 is movable relative to the pusher 146 and movable relative to the slides (not shown) (and therefore by extension the two slide rails 136 as well). In the illustrated embodiment, the fluid connector mount 152 is free floating. This allows the fluid connector mount 152 to move in generally any direction, which facilitates the alignment of the fluid connectors 154 with their corresponding exterior fluid connector ports 32 of the sterilization container 12. Specifically, the fluid connector mount 152 and the fluid connectors 154 are freely moveable relative to the sterilization chamber 102 (e.g., the slide rails 136) along or within (broadly, parallel to) an imaginary reference plane RP (FIG. 14) that is normal to the longitudinal axis LA. This allows the fluid connectors 154 to freely move vertically and/or laterally to align with the corresponding exterior fluid connector ports 32 of the sterilization container 12. In the illustrated embodiment, the fluid connector mount 152 is supported by the pusher 146. The fluid connector mount 152 includes two oversized openings 156 sized and shaped to receive a forward portion of each of the slide bearings. The openings 156 have a diameter larger than the diameter of the forward portion of each slide bearing. Preferably, the diameter of the openings 156 is about 5/32 inch (about 4 mm) larger than that diameter of the forward portion of each of the slide bearings. The oversized openings 156 permit the fluid connector mount 152 and the fluid connectors 154 to move along the reference plane RP to align with the exterior fluid connector ports 32 of the sterilization container 12. The forward portion of each slide bearing includes a flange or shoulder that engages a rear surface or face of the fluid connector mount 152 to push the fluid connector mount along the longitudinal axis LA from the retracted position to the extended position.

The container connector assembly 134 may include two clips or stops (not shown), such as two C-clips, which retain the fluid connector mount 152 on the forward portions of the slide bearings of the pusher 146. Thus, the fluid connector mount 152 is generally restrained from moving longitudinally relative to the pusher 146 but is free to move vertically and laterally relative to the pusher 146. The container connector assembly 134 also includes two clips or stops, such as two C-clips (not shown), adjacent the rear end of each slide to retain the pusher 146 on the slides.

The initial alignment guides 140 can move (e.g., longitudinally move) with respect to the fluid connectors 154. This relative movement assists in connecting the fluid connectors 154 to the container 12, as described in more detail below. In one embodiment, the initial alignment guides 140 may be biased in a forward direction (e.g., toward the extended position). In one embodiment, the container connector assembly 134 includes two springs (not shown), such as coil springs, biasing the initial alignment guides 140 toward the extended position. In one embodiment, each spring surrounds one of the slides defining the initial alignment guide 140, with one end of the spring engaging the flange of the initial alignment guide 140 and the other end of the spring engaging the fluid connector mount 152 and/or clip.

The container connector assembly 134 includes two (broadly, at least one) final alignment guides or pins 162. Each final alignment guide 162 of the container connector assembly 134 is configured to engage the sterilization container 12 to align the fluid connectors 154 and the exterior fluid connector ports 32 with one another. Specifically, each final alignment guide 162 of the container connector assembly 134 is configured to mate with (e.g., be inserted into) a corresponding one of the final alignment guides 50 of the sterilization container 12. In the illustrated embodiment, each final alignment guide 162 includes a forward end portion arranged to be inserted into the blind hole of the corresponding one of the final alignment guides 50 of the sterilization container 12. The forward end portion may include a conical tip.

In one embodiment, the sterilization apparatus 100 is configured to determine whether or not the container connector assembly 134 is mated with the sterilization container 12. In the illustrated embodiment, each final alignment guide 162 may include a fluid passageway extending longitudinally therethrough and a rearward conduit connection portion configured to be connected to a flexible fluid conduit (not shown) of the sterilization apparatus 100. The forward end portion of each final alignment guide 162 may include a seal, such as an O-ring, that engages the interior surface of the final alignment guide 50 of the sterilization container 12 that defines the blind hole to form a fluid tight seal between the final alignment guide of the sterilization apparatus 100 and the sterilization container 12. This allows the sterilization apparatus 100 to draw a vacuum through the final alignment guide 162 of the container connector assembly 134 via the fluid passageway. If the sterilization apparatus 100 can draw a vacuum through the final alignment guide 162, then the sterilization apparatus can confirm the container connector assembly 134 is sufficiently mated with the sterilization container 12. The sterilization apparatus 100 can include the appropriate pump (for drawing the vacuum), sensor (for detecting the vacuum) and plumbing (not shown) fluidly connected to the final alignment guide 162 of the container connector assembly 134 for drawing and detecting the vacuum. Other ways of determining whether or not the container connector assembly is mated with the sterilization container, such as sensors, buttons, etc., are within the scope of the present disclosure.

The sterilization apparatus 100 includes a prime mover 164 operatively coupled to the container connector assembly 134 to move the container connector assembly between the retracted and extended positions. In particular, the prime mover 164 is operatively coupled to the fluid connectors 154 and configured to move the fluid connectors between the retracted and extended positions. In the illustrated embodiment, the prime mover 164 is a linear actuator, although other types of prime movers (e.g., electric motors) are within the scope of the present disclosure. A drive train interconnects the container connector assembly 134 and the prime mover. The drive train includes a drive shaft 166 connected at one end to the linear actuator and to the pusher 146 (e.g., the push plate 148) at the other end (broadly, the drive train includes the pusher). The drive shaft 166 extends through a slide bearing (not shown) secured to the rear wall 116. The sterilization apparatus 100 includes a switch assembly 170 including first and second switches (not shown), such as limit switches. The first switch is positioned such that it is engaged by the drive train when the container connector assembly 134 is in the retracted position. Similarly, the second switch is positioned such that it is engaged by the drive train when the container connector assembly 134 is in the extended position. By actuating one of the switches, the sterilization apparatus 100 can know when the container connector assembly 134 is in either the retracted position or the extended position and stop the prime mover 164 from continuing to operate, thereby positioning the container connector assembly 134 is in either the retracted position or the extended position.

Referring to FIGS. 12-14, the operation of the container connector assembly 134 as the container connector assembly moves between the retracted and extended positions will now be described. FIG. 12 shows the container connector assembly 134 at rest in the retracted position. Starting at the retracted position, the linear actuator 164 holds the pusher 146, and therefore the fluid connector mount 152 and fluid connectors 154, in the longitudinal position (i.e., the retracted position) shown in FIG. 12. The springs may bias the initial alignment guides 140 forward, toward the extended position. In one embodiment, the clips (broadly, stops) attached at the rear end of each slide engage the rear of the pusher 146, thereby position the initial alignment guides 140 in the position shown in FIG. 12. With the container connector assembly 134 in the retracted position, the sterilization chamber 102 is ready to receive the sterilization container 12. A user opens the door 118 and inserts the sterilization container 12 into the interior 122. As the sterilization container 12 is inserted into the sterilization chamber 102, the guide rails may guide the sterilization container vertically and/or laterally to position the sterilization container in a rough alignment with the container connector assembly 134. The sterilization container 12 is moved rearward in the interior 122 until the sterilization container contacts the container connector assembly 134 and/or a rear stop 176 (FIG. 9) (broadly, at least one stop) at the rear of the interior. After the sterilization container 12 is positioned in the interior 122 of the sterilization chamber 102, the door 118 is closed. It is appreciated that before the sterilization container 12 is placed in the sterilization chamber 102, the one or more articles are placed in the interior 16 of the sterilization container and each connected to respective interior fluid connector ports 34 via the article connectors.

After the door 118 is closed, the container connector assembly 134 moves toward the extended position. To move the container connector assembly 134 toward the extended position, the linear actuator 164 moves the pusher 146 forward. As the pusher 146 moves forward, the pusher 146 pushes or moves the fluid connector mount 152 forward as well. In one embodiment, the springs continue to bias the initial alignment guides 140 (e.g., slides) in the forward direction as well. Accordingly, as the pusher 146 moves forward, so do the initial alignment guides 140, the springs keeping the clips engaged with the pusher. The slides slide forward along the slide rails 136. At this instance in the operation, the pusher 146, the fluid connector mount 152, the fluid connectors 154, the initial alignment guides 140, the slides, and the final alignment guides 162 all move together in the forward direction. Eventually, the initial alignment guides 140 engage the sterilization container 12. As the initial alignment guides 140 move forward, the initial alignment guides of the container connector assembly 134 mate with the initial alignment guides 48 of the sterilization container 12. The tip of each initial alignment guide 140 of the container connector assembly 134 is inserted to the blind hole of the corresponding initial alignment guide 48 of the sterilization container 12. During this process, engagement between the initial alignment guides 140, 48 may move the sterilization container 12 vertically and/or laterally to position the sterilization container 12 relative to the container connector assembly 134. The initial alignment guides 140 of the container connector assembly 134 continue to be inserted into the corresponding initial alignment guides 48 until the flanges of the initial alignment guides 140 engage the sterilization container 12.

With the flanges of the initial alignment guides 140 engaged with the sterilization container 12, the flanges push the sterilization container forward against the stop 130 on the door 118. The stop 130 limits or blocks the continued forward movement of the sterilization container 12. At this time, the initial alignment guides 140 have reached their forward most position (i.e., are in the extended position) and are inhibited from further forward movement by the sterilization container 12 and stop 130. The initial alignment guides 140 (and slides) now do not move relative to the slide rails 136. However, the linear actuator 164 continues to move the pusher 146 in the forward direction. The linear actuator 164 applies a sufficient amount of force to overcome the biasing force of any springs, such that the springs begin to compress. At this instance in the operation, the pusher 146, the fluid connector mount 152, the fluid connectors 154, and the final alignment guides 162 still move together in the forward direction, relative to the initial alignment guides 140 (e.g., along the slides). In one embodiment, as the pusher 146 continues to move forward, the slide bearings slide along the slides. Eventually, the final alignment guides 162 engage the sterilization container 12. As the final alignment guides 162 move forward, the final alignment guides of the container connector assembly 134 mate with the final alignment guides 50 of the sterilization container 12. Each final alignment guide 162 of the container connector assembly 134 is inserted to the blind hole of the corresponding final alignment guide 50 of the sterilization container 12. During this process, engagement between the final alignment guides 162, 50 may move the fluid connector mount 152 and fluid connectors 154 vertically and/or laterally along the reference plane RP (relative to the pusher 146 and the sterilization container 12) to align the fluid connectors with the exterior fluid connector ports 32 of the sterilization container 12. As the final alignment guides 162 of the container connector assembly 134 are inserted into the corresponding final alignment guides 50, the final alignment guides of the container connector assembly 134 form a seal with the final alignment guides 50 of the sterilization container 12, thereby allowing the vacuum to be created as described above.

After the final alignment guides 162, 50 mate with each other, and as the linear actuator continues to move the pusher 146 forward, the fluid connectors 154 are inserted into their corresponding exterior fluid connector ports 32 of the sterilization container 12 (FIG. 14). The pusher 146 continues to move forward, pushing the fluid connectors 154 toward and into engagement with the valves 38 (broadly, the valve actuator 46) associated with each exterior fluid connector port 32. This engagement opens the valve 38, as described herein, thereby creating an open fluid pathway between the sterilization container 12 (e.g., the interior 16) and the sterilization apparatus 100 (e.g., the fluid connector 154). After this engagement, the linear actuator 164 stops moving the pusher 146 forward. At this time, the container connector assembly 134 is now in the extended position (FIG. 13). The sterilization apparatus 100 can now move (e.g., draw) sterilization fluid into the sterilization container 12 from the interior 122 of the sterilization chamber 102, through the articles, through the open valves 38, and into the fluid connectors 154 to sterilize the articles.

After the sterilization process is complete, the movement of the container connector assembly 134 is generally reversed to disconnect the container connector assembly from the sterilization container. The linear actuator 164 moves the pusher 146 in a rearward direction. The pusher 146 moves the fluid connector mount 152, the fluid connectors 154 and the final alignment guides 162 in the rearward direction as well. As the fluid connector mount 152 moves rearward, the fluid connectors 154 disengage or withdraw from exterior fluid connector ports 32. Likewise, the final alignment guides 162 of the container connector assembly 134 disengage or withdraw from the final alignment guides 50 of the sterilization container 12. The pusher 146, fluid connector mount 152, the fluid connectors 154 and the final alignment guides 162 may move rearward relative to the initial alignment guides 140 (e.g., along the slides) until the pusher 146 engages the clips at the rear ends of the slides. Any springs expand as the pusher 146 moves rearward to keep the initial alignment guides 150 (e.g., slides) in generally as stationary position until the clips are engaged by the pusher 146. With the pusher 146 continuing to move rearward, the pusher engages the clips at the rear end of the slides, thereby moving the initial alignment guides 140 and slides rearward along the slide rails 136. At this instant, the pusher 146, the fluid connector mount 152, the fluid connectors 154, the final alignment guides 162, and the initial alignment guides (e.g., the slides) all move rearward together. As the initial alignment guides 140 move rearward, the initial alignment guides disengage or withdraw from the initial alignment guides 48 of the sterilization container 12. The pusher 146, the fluid connector mount 152, the fluid connectors 154, the final alignment guides 162, and the initial alignment guides 140 continue to move rearward until they reach the retracted position, as which point the linear actuator 164 stops moving, thereby holding the container connector assembly 134 in the retracted position. The door 118 can now be opened by the user and the sterilization container 12 removed from the sterilization chamber 102, containing sterilized articles. If the articles are to be used right away, the lid 20 of the sterilization container 12 can be opened to remove the sterilized articles from the container. If the articles don't need to be used right away, the sterilization container 12 can be stored until such time as when the articles are needed. As mentioned herein, the sterilization container 12 will keep the articles sterilized while the articles are being stored.

As is apparent, in the sterilization system 10 described above the fluid connectors 154 generally move relative to the sterilization container 12 to fluidly couple the sterilization container to the sterilization apparatus 100. In another embodiment, the sterilization container can generally move relative to the fluid connectors to fluidly couple the sterilization container to the sterilization apparatus. In this embodiment, the sterilization container and the sterilization apparatus may be generally the same as described above, except that instead of or in addition to movable container connector assembly 134, the sterilization apparatus may include a container mover that moves the container toward the fluid connectors to fluidly couple the fluid connectors to the container. The container mover may move the sterilization container between a first or disengaged position, where the sterilization container is not fluidly coupled to the fluid connectors, and a second or engaged position, where the sterilization container is fluidly coupled to the fluid connector. In one example, the container mover includes a pair of jaws that releasably engage opposite sides of the sterilization container and a prime mover (e.g., an electric motor, linear actuator, etc.) that moves the jaws (and sterilization container gripped thereby) between the engaged and disengaged positions. In another example, the container mover comprises a pusher operatively coupled to a prime mover. The pusher is arranged to engage the sterilization container and push the sterilization container rearward toward the fluid connectors (e.g., toward the engaged position). It is understood the sterilization apparatus may include both (broadly, at least one of) the movable container connector assembly 134 and the container mover.

It is apparent and understood that the elements, features, and/or teachings set forth in each embodiment disclosed herein are not limited to the specific embodiment(s) the elements, features, and/or teachings are described in. Accordingly, it is apparent and understood that the elements, features, and/or teachings described in one embodiment may be applied to one or more of the other embodiments disclosed herein.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

Modifications and variations of the disclosed embodiments are possible without departing from the scope of the invention defined in the appended claims. For example, where specific dimensions are given, it will be understood that they are exemplary only and other dimensions are possible. As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A sterilization container for use with a sterilization apparatus for sterilizing an article, the sterilization container comprising:
a body defining an interior configured to receive the article;
an exterior fluid connector port facing an environment surrounding the body, the exterior fluid connector port fluidly connectable to a first fluid connector of the sterilization apparatus;
an interior fluid aperture facing the interior and in fluid communication with the interior of the body, the interior fluid aperture configured to be fluidly connected to the article, the interior fluid aperture being fluidly coupled to the exterior fluid connector port; and
a valve fluidly disposed between the exterior fluid connector port and the interior fluid aperture, the valve including a valve member movable to an open position in which the valve permits fluid flow between the exterior fluid connector port and the interior fluid aperture and to a closed position in which the valve blocks fluid flow between the exterior fluid connector port and the interior fluid aperture, the valve arranged to be engaged by the sterilization apparatus to move the valve member toward the open position to permit fluid flow between the first fluid connector and the article when the sterilization container is used with the sterilization apparatus and when the article is fluidly connected to the interior fluid aperture.

2. The sterilization container of claim 1, wherein the valve is arranged to be engaged by the first fluid connector of the sterilization apparatus to move the valve member to the open position.

3. The sterilization container of claim 1, wherein the valve member is biased toward the closed position.

4. The sterilization container of claim 1, wherein the exterior fluid connector port defines an exterior port aperture sized and shaped to receive the first fluid connector.

5. The sterilization container of claim 1, further comprising an interior fluid connector port, the interior fluid connector port defining the interior fluid aperture, the interior fluid connector port configured to be fluidly connected to an article fluid connector associated with the article to fluidly connect the article to the interior fluid aperture.

6. The sterilization container of claim 1, wherein the body includes an initial alignment guide configured to be engaged by the sterilization apparatus to initially position the sterilization container relative to the sterilization apparatus when the sterilization container is used with the sterilization apparatus.

7. The sterilization container of claim 6, wherein the body includes a final alignment guide configured to be engaged by the sterilization apparatus to align the exterior fluid connector port and the first fluid connector with one another when the sterilization container is used with the sterilization apparatus.

8. The sterilization container of claim 1, wherein the article is a first article, the exterior fluid connector port is a first exterior fluid connector port, the interior fluid aperture is a first interior fluid aperture, and the valve is a first valve, the sterilization container further comprising:
a second exterior fluid connector port facing the environment surrounding the body, the second exterior fluid connector port fluidly connectable to a second fluid connector of the sterilization apparatus;
a second interior fluid aperture facing the interior of the body and in fluid communication with the interior of the body, the second interior fluid aperture configured to be fluidly connected to a second article, the second interior fluid aperture being fluidly connected to the second exterior fluid connector port; and
a second valve fluidly disposed between the second exterior fluid connector port and the second interior fluid aperture, the second valve including a valve member movable to an open position in which the second valve permits fluid flow between the second exterior fluid connector port and the second interior fluid aperture and to a closed position in which the second valve blocks fluid flow between the second exterior fluid connector port and the second interior fluid aperture, the second valve arranged to be engaged by the sterilization apparatus to move the valve member of the second valve toward the open position to permit fluid flow between the second fluid connector and the second article when the sterilization container is used with the sterilization apparatus and when the second article is fluidly connected to the second interior fluid aperture.

9. The sterilization container of claim 1, further comprising a sterilant permeable membrane configured to permit a sterilant in the environment surrounding the body to pass therethrough and into the interior of the body.

10. The sterilization container of claim 1, wherein the body includes a box and a lid attachable to the box.

11. A sterilization apparatus for sterilizing an article within a sterilization container, the sterilization apparatus comprising:
a sterilization chamber defining an interior sized and shaped to receive the sterilization container; and
a container connector assembly configured to fluidly connect the sterilization container to the sterilization apparatus, the container connector assembly including a fluid connector movable between a retracted position and an extended position, the fluid connector being configured to engage the sterilization container when the fluid connector moves from the retracted position toward the extended position to fluidly connect the fluid connector to the sterilization container.

12. The sterilization apparatus of claim 11, further comprising a prime mover operatively coupled to the fluid connector and configured to move the fluid connector between the retracted position and the extended position.

13. The sterilization apparatus of claim 11, wherein the container connector assembly includes a pusher configured to move the fluid connector from the retracted position toward the extended position.

14. The sterilization apparatus of claim 13, wherein the pusher is slideably mounted on at least one rail.

15. The sterilization apparatus of claim 13, wherein the container connector assembly includes a fluid connector mount, the fluid connector mounted to the fluid connector mount, the pusher operatively connected to the fluid connector mount to move the fluid connector from the retracted position toward the extended position.

16. The sterilization apparatus of claim 15, wherein the container connector assembly includes an initial alignment guide configured to engage the sterilization container to initially position the sterilization container relative to the sterilization chamber.

17. The sterilization apparatus of claim 16, wherein the fluid connector mount includes a final alignment guide configured to engage the sterilization container to align the fluid connector and a fluid connector port of the sterilization container with one another.

18. The sterilization apparatus of claim 11, wherein the fluid connector moves along a longitudinal axis between the retracted position and the extended position, wherein the fluid connector is freely moveable relative to the sterilization chamber along an imaginary plane that is normal to the longitudinal axis.

19. The sterilization apparatus of claim 11, wherein the sterilization chamber includes a stop arranged to engage the sterilization container and to limit movement of the sterilization container as the fluid connector moves from the retracted position to the extended position.

20. The sterilization apparatus of claim 11, in combination with the sterilization container, such as the sterilization container of claim 1.

21. The sterilization apparatus of claim 20, wherein the sterilization container is configured to maintain the sterility of the article after sterilization by automatically sealing itself as the container connector assembly moves toward the retracted position and disengages the sterilization container.

22. A sterilization system for sterilizing an article, the sterilization system comprising:
a sterilization container configured to receive the article;
a sterilization chamber defining an interior sized and shaped to receive the sterilization container; and
a fluid connector configured to fluidly connect the sterilization container to the sterilization apparatus;
wherein at least one of the fluid connector or the sterilization container is configured to move toward the other of the fluid connector or the sterilization container to fluidly connect the fluid connector and the sterilization container to one another.
